(19) United States
(12) Patent Application Publication
(10) Pub. No.: US 2013/0035379 A1
(43) Pub. Date: Feb. 7, 2013

(54) Compositions for Inhibiting Protine-Directed Protein Kinase Fa/Glycogen Synthesis Kinase 3 Alpha and Use Thereof

(71) Applicants: Shiaw-Der Yang, Hsinchu (TW); Hsiao-Hui Fu, Hsinchu (TW)

(72) Inventors: Shiaw-Der Yang, Hsinchu (TW); Hsiao-Hui Fu, Hsinchu (TW)

(73) Assignee: NATIONAL TSING HUA UNIVERSITY, Hsinchu (TW)

(21) Appl. No.: 13/473,226

(22) Filed: May 16, 2012

(30) Foreign Application Priority Data
Aug. 1, 2011 (TW) 100127229

(51) Int. Cl. A61K 31/353; A61P 35/00; A61P 35/02; C07D 311/62

(52) U.S. Cl. 514/456; 549/403

(57) ABSTRACT

The invention provides a composition for inhibiting proline-directed protein kinase F_{A}/glycogen synthase kinase 3α (PDPK F_{A}/GSK-3α), experiments verified that the composition can effectively inhibit the PDPK F_{A}/GSK-3α. In addition, inhibition of PDPK F_{A}/GSK-3α can improve the clinical efficacy of cancer treatment and cure rate.

### CROSS-REFERENCE TO RELATED APPLICATIONS

This Non-provisional application claims priority under 35 U.S.C. §119(a) on Patent Application No(s). 100127229 filed in Taiwan, Republic of China, Aug. 1, 2011, the entire contents of which are hereby incorporated by reference.

### FIELD OF THE INVENTION

The present invention relates to a composition for inhibiting proline-directed protein kinase FA/glycogen synthase kinase 3 alpha (PDPK FA/GSK-3α).

### BACKGROUND OF THE INVENTION

Oligomer procyanidins (OPC) is a family of bioflavonoids, a type of polyphenols. This substrate is generally exhibited in plants, and is often exhibited in fruits, nuts, seeds, flowers, bark and vegetables, especially high in grape seed (Wilska, 1996; Pekic et al., 1998; Yamakoshi et al., 1999; Bruno et al, 2000; Yamakoshi, 2002; Shao et al., 2003) and pine bark extract. Recently, all kinds of studies indicate there are anti-oxidant components in bioflavonoids, which have protection functions of anti-cancer, anti-allergy, etc.

In early stage, the OPC was used as drug, such as: Endotelon for treating microvascular circulation abnormality, besides, enhancing immune system and circulation system. OPC is general used for treating high blood pressure, heart disease prevention, vascular disease and decreasing the diabetic retinopathy in abroad (Motoaki et al., 1999). In addition, there are many applications in beauty products such as impediment to the injury of UV and reduction the break of collagen and elastic fibril in the skin, in order to prevent the wrinkle caused by skin collapse and to protect skin from aging, and then to achieve the effect of anti-aging (Garbacki et al., 2002). OPC is usually used in anti-oxidation, mostly includes anti-peroxidation and free radical cleaning, especially shows the apparent effect on free radical cleaning. Besides, OPC has effect on inhibirion of bacteria and cancer cell proliferation, and anti-inflammation (Madhavin et al., 1998; Hassan et al., 2000; Wollgast et al., 2000).

OPC is not a single compound, it is composed of a series of compounds which includes high and low molecule weight compounds, wherein the low molecule weight compounds comprise catechin and epicatechin, dimer (procyanidi series B), polyhydroflavan-3-ol, trimmer (procyanidin C series); the high molecule weight compounds comprise tannins (condensed tannins) and polymers (polyhydroflavan-3-ol). However, every monomer is linked by carbon-carbon bound (Balde et al., 1995; de Bruyne et al., 1999; Bennie et al., 2000; Gabetta et al., 2000).

Taking grape seed for example, the chemical structure is mainly constructed by flavan-3-ol, wherein the most important component is oligomer procyanidins (OPC). The OPC can be classed as monomeric, oligomeric and polymeric proanthocyanidins by degree of polymerization (DP). And the feature of OPC is mostly related to its structure, especially related to the degree of polymerization (Haslam, 1974; Robichaud et al., 1990; Rigaud et al., 1993; Hor et al., 1996; Baoshan et al., 1998; Le Roux et al., 1998). The basic monomer of OPC is flavan-3-ol, which is called as catechin or the other stereochemical isomer—epicatechin. The OPC itself has little activity (Poorter et al., 1986). However, when they combine as dimers or trimmers, which is an oligomer, so called as OPC or Procyanidin (Foo et al., 1999), which has high biological activity. Two monomers are called as dimers, and three monomers are called as trimmers, and four monomers are called as tetramers and so forth, if the polymer is composed of more than four monomers, it is called polymeric proanthocyanidins. However, the OPC in the grape seed are different types of compounds which are composed of 2˜17 monomers (Prieur et al., 1994).

PDPK FA was originally identified as a specific membrane and cytoplasmic activating factor A of ATP.Mg-dependent protein phosphatase but has subsequently been characterized as a multisubstrate/multifunctional proline-directed protein kinase (PDPK) possibly associated with human cancers. In recent years, the immunohistochemical study revealed that PDPK FA was not only highly expressed in histologically conventional tumor cells but also aberrant accumulated in a signaling interplay network (named as lethal system) preferably epithelial-mesenchymal transition (EMT) or tumor-stroma coevolutional communication (TSCC) associated with aberrant expression of PDPK FA in other tumor-associated cells such as bone marrow-derived cell (BMDC) or cancer-associated fibroblast (CAF) or circulating tumor cell (CTC) or circulating tumor stem cell (CTSC) or epithelial tumor cell (ETC) or epithelial tumor stem cell (ETSC) or hematopoietic stem/progenitor cell (HSPC) or mesenchymal stem cell (MSC) or mesenchymal tumor cell (MTC) or mesenchymal tumor stem cell (MTSC) or tumor-associated macrophage (TAM) useful in detecting micrometastasis and PDPK FA in bone marrow-derived stem cell (BMDSC; named as lethal cell) is useful for monitoring disease status and therapy responses in various types of cancer patients regardless of the etiological origin of the cancer. Furthermore, PDPK F_{A}/GSK-3α in bone marrow cell (BMC; named as unhealthy BMC) has been established as the diagnostic indicator for predicting disease predisposition from very early-stage aberrant wound healing, chronic inflammation and immune disorder towards end-stage fibrotic disease or neoplasia in cancer-free human subjects and has been established as a master integrator of stresses and inflammation responses essential for development and progression of certain illnesses (vicious cycle).

As used in the present invention, the term “unhealthy BMC” refers to a cell preferably a BMC associated with aberrant expression of PDPK F_{A}/GSK-3α. Please check our previous U.S. patent application Ser. No. 12/495,967 for further information. And the term “lethal cell” refers to a BMDSC associated with an aberrant intracellular accumulation of PDPK F_{A}/GSK-3α. Please check our previous U.S. patent application Ser. No. 12/553,035 for further information. And the term “lethal system” refers to a signaling interplay network preferably epithelial-mesenchymal transition (EMT) or tumor-stroma coevolutional communication (TSCC) associated with aberrant expression of NFA in said marker cell. Please check our previous U.S. patent application Ser. No. 13/186,100 for further information. Moreover, the term “vicious cycle” indicates pathophysiological condition induced by PDPK F_{A}/GSK-3α related cytokines expression when cells are under pressure and inflammation state. Please check our previous U.S. patent application Ser. No. 13/166,715 for further information.

However, due to high sequence homology in kinase domain, PDPK FA was regarded as a subtype of GSK 3 (glycogen synthase kinase 3), and renamed as GSK-3α. Although GSK 3/GSK-3β and PDPK FA/GSK-3α have long been regarded as two closely-related signaling molecule, albeit structurally-similar in kinase domain, they are not functionally-equivalent or -redundant as previously conceived in drosophila and rodents when based on human clinical studies as demonstrated in this application. Moreover, intensive study and most attention have focused on GSK-3β but entitled GSK3 without further specifications in many areas of research and suppression of this kinase may cause tumorigenesis which raises a serious issue concerning how to treat diabetes without causing cancer. As a result, the unique role of PDPK FA has been overlooked for more than a decade and the current development of GSK-3 inhibitor is restricted in inhibiting activity of GSK-3α/β. The specific inhibitors of PDPK FA/GSK-3α have never been produced. Thus, to develop an inhibitor of PDPK FA/GSK-3α is an urgent case.

### SUMMARY OF THE INVENTION

In the present invention, a composition for inhibiting proline-directed protein kinase F_{A}/glycogen synthase kinase 3α (PDPK FA/GSK-3α) which composition comprises an oligomer procyanidins (OPCs).

Accordingly, the oligomer procyanidins is extracted by grape, grape skin, grape seed, pine bark, bark of lemon tree, peanuts, berries, sorghum, apple, coco bean, cherry, strawberry, hops, salix, cinnamon, tea leaf, Wax Tree, Pinto Bean, red kidney beans, hazelnut, or walnut. The composition further comprising a pharmaceutically acceptable carrier. And the pharmaceutical acceptable carrier is excipient, diluents, thickeners, filler, binder, disintegrants, lubricant, oil or non-oil base, surfactant, suspending agent, gelling agent, adjuvant, anti-corrosive agent, anti-oxidant, stabilizer, coloring agent or flavor.

Another purpose of the present invention is a method for treating an unhealthy state in a subject through inhibiting PDPK FA/GSK-3α expression by administering an effective amount of OPC

Accordingly, the unhealthy state is a human who is free of diagnosable cancer and has an unhealthy BMC. And the unhealthy BMC is a BMC associated with an aberrant intracellular accumulation of PDPK FA/GSK-3α. Wherein the BMC is selected from the group consisting of stem cell, bone marrow derived cell (BMDC), mesenchymal stem cell (MSC), hematopoietic stem and progenitor cell (HSPC), cancer stem cell (CSC), fibrocyte, macrophage, fibroblast, myoblasts, mesenchymal cell, and the cell analogue. Wherein the aberrant intracellular accumulation of PDPK FA/GSK-3α expression is in cytoplasmic and/or nuclear expression of PDPK FA/GSK-3α protein, mRNA or DNA level.

Accordingly, the unhealthy state is a human who is free of diagnosable cancer and has a vicious cycle. And the vicious cycle is stresses and/or inflammations responses associated with an aberrant accumulation of PDPK FA/GSK-3α.

Another purpose of the present invention is a method for treating a cancer patient through inhibiting PDPK FA/GSK-3α expression cell by administering an effective amount of OPC.

For the purpose, the cancer patient has a lethal cell. And the lethal cell is a BMDSC associated with an aberrant intracellular accumulation of PDPK FA/GSK-3α.

For the purpose, the cancer patient has a lethal system. And the lethal system is a signaling interplay network associated with aberrant expression of PDPK FA/GSK-3α in marker cell. And the signaling interplay network is selected from the group consisting of epithelial-mesenchymal transition (EMT) and tumor-stroma coevolutional communication (TSCC). Wherein the marker cell is selected from the group consisting of bone marrow-derived cell (BMDC), carcinoma-associated fibroblasts (CAF), circulating tumor cell (CTC), circulating cancer stem cell (CTSC), epidermal tumor cell (ETC), epidermal tumor stem cell (ETSC), cancer stem cell (CSC), tumor proliferate cell (TPC), hematopoietic stem and progenitor cell (HSPC), mesenchymal stem cell (MSC), mesenchymal tumor cell (MTC), mesenchymal tumor stem cell (MTSC), tumor-associated macrophage (TAM), and myeloid cell.

Accordingly, the cancer is selected from a group consisting of bladder cancer, breast cancer, brain cancer, cancer of biliary duct, uterine cancer, esophageal cancer, stomach cancer, hepatic cancer, lung cancer, oral cancer, ovarian cancer, pancreatic cancer, prostate cancer, kidney cancer, leukemia and lymphoma.

### BRIEF DESCRIPTION OF THE DRAWINGS

FIG. 1 depicts the aberrant cytoplasmic and/or nuclear protein expression patterns of PDPK F_{A}/GSK-3α in BMC by co-immunohistochemical staining of a rare population of CD34⁺ and CD34⁻ stem/progenitor cells associated with very strong cytoplasmic and nuclear expressions (>3+) of PDPK F_{A}/GSK-3α and their possible derivatives with moderate-strong cytoplasmic and/or nuclear protein expression (2+ to 3+) of PDPK F_{A}/GSK-3α in bone marrow of poor outcome leukemia patients. (A). Positive pattern co-staining with CD34, (B). Negative pattern co-staining with CD34.

FIG. 2 depicts the representative cytoplasmic and/or nuclear protein expression pattern of PDPK F_{A}/GSK-3α in poor outcome stage I tumor stromas by co-immunohistochemical staining of a rare population of CD34⁺/vimentin⁻, CD34⁺/vimentin⁺ and CD34⁻/vimentin⁺ cells associated with very strong cytoplasmic and/or nuclear protein expression (>3+) of PDPK F_{A}/GSK-3α and a relatively large population of their derivatives vimentin⁺ fibroblasts/mesenchymal cells, CD68⁺ macrophages and α-smooth muscle actin (α-SMA)⁺ myofibroblast associated with moderate to strong cytoplasmic and/or nuclear protein expression (2+ to 3+) of PDPK F_{A}/GSK-3α. (A). Positive pattern co-staining with CD34, (B). Negative pattern co-staining with CD34, (C). Positive pattern co-staining with vimentin, (D). Negative pattern co-staining with vimentin, (E). Positive pattern co-staining with CD68, (F). Negative pattern co-staining with CD68, (G). Positive pattern co-staining with α-SMA, (H). Negative pattern co-staining with α-SMA.

FIG. 3 establishes PDPK F_{A}/GSK-3α as an early unhealthy signal of a specific unique subset of BMC by co-immunocytochemical staining of CD34⁺ fibrocytes-like cells associated with strong cytoplasmic and/or nuclear protein expression of PDPK F_{A}/GSK-3α in peripheral blood of children from cancer family or suffering from repeated cycles of inflammations. (A). Positive pattern, (B). Negative pattern.

FIG. 4 further establishes PDPK F_{A}/GSK-3α as a very early unhealthy signal of a specific unique subset of BMC by co-immunocytochemical staining of CD34⁺ hematopoietic stem/progenitor cells expressing cytoplasmic and/or nuclear PDPK F_{A}/GSK-3α in umbilical cord blood of cancer family child suffering from immune system disorder characteristic of Kawasaki syndrome.

FIG. 5 depicts the disease-free survival of various types of cancer patients with respect to lethal cell.

FIG. 6 depicts the Kaplan-Meier disease-free survival (DFS) curve of various types of patients with very early stage I tumors with respect to the presence and absence of the recruited BMC expressing cytoplasmic and/or nuclear protein expression of PDPK F_{A}/GSK-3α within stroma tissues of breast, bile duct, colorectum, cervix, esophagus, stomach, liver, lung, oral cavity, ovary, pancreas, prostate and kidney.

FIG. 7 demonstrates (A) the PDPK F_{A}/GSK-3α expression type before using OPC, and (B) the inhibition effect of PDPK F_{A}/GSK-3α expression after using OPC in patients with acute leukemia.

FIG. 8 demonstrates (A) the PDPK F_{A}/GSK-3α expression type before using OPC, and (B) the inhibition effect of PDPK F_{A}/GSK-3α expression after using OPC in patients with lymphoma.

FIG. 9 demonstrates (A) the PDPK F_{A}/GSK-3α expression type before using OPC, and (B) the inhibition effect of PDPK F_{A}/GSK-3α expression after using OPC in patients with kidney cancer.

FIG. 10 demonstrates (A) the PDPK F_{A}/GSK-3α expression type before using OPC, and (B) the inhibition effect of PDPK F_{A}/GSK-3α expression after using OPC in patients with uterine sarcoma.

FIG. 11 demonstrates (A) the PDPK F_{A}/GSK-3α expression type before using OPC, and (B) the inhibition effect of PDPK F_{A}/GSK-3α expression after using OPC of peripheral blood in systematic unhealthy children from cancer family.

### DETAILED DESCRIPTION OF THE INVENTION

### Term Definition

For clarity of disclosure, and not by way of limitation, the detailed description of the invention is divided into the subsections that follow.

Unless defined otherwise, all technical and scientific terms used herein have the same meaning as is commonly understood by one of ordinary skill in the art to which this invention belongs. All patents, applications, published applications and other publications, and Genebank Accession numbers referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used in the present invention, the term “PDPK F_{A}/GSK-3α” indicates multisubstrate/multi-functional proline-directed protein kinase F_{A}, which also known as glycogen synthase kinase 3α. (Woodgett, EMBO J, 1990, 9:2431-2438; Yang, Curr Cancer Drug Targets, 2004, 4:591-596). The NCBI number of this protein is AAD11986 and AAH27984.

As used herein, the term “unhealthy BMC” refers to a cell preferably a BMC associated with aberrant expression of PDPK F_{A}/GSK-3α. Please check our previous U.S. patent application Ser. No. 12/495,967 for further information.

As used herein, the term “lethal cell” refers to a BMDSC associated with an aberrant intracellular accumulation of PDPK F_{A}/GSK-3α. Please check our previous U.S. patent application Ser. No. 12/553,035 for further information.

As used herein, the term “lethal system” refers to a signaling interplay network preferably epithelial-mesenchymal transition (EMT) or tumor-stroma coevolutional communication (TSCC) associated with aberrant expression of NFA in said marker cell. Please check our previous U.S. patent application Ser. No. 13/186,100 for further information.

As used in the present invention, the term “vicious cycle” indicates pathophysiological condition induced by PDPK F_{A}/GSK-3α related cytokines expression when cells are under pressure and inflammation state. Please check our previous U.S. patent application Ser. No. 13/166,715 for further information.

As used in the present invention, the term “marker cell” indicates a cell can be identified by a cell marker, includes but not limited to CD34, CD90, CD68, vimentin, FSP-1 or αSMA.

Preferably, the individual is in “unhealthy state” indicateds the individual has an unhealthy BMC or the individual has a vicious cycle induced pathophysiological clinical symptom.

Unless otherwise indicated, all terms, which include embodiments and conventional techniques of biochemical and clinical pathological technology, disclosed herein have the same meaning within the knowledge of those skilled in the art.

The present invention provides “cancer prevention, disease predisposition prevention, metastasis prevention and vicious cycle prevention by OPC induced PDPK FA/GSK-3α inhibition effectively” which can be understood by the description of the following embodiments, enable a person of ordinary skill in the art to make and use the present invention. However, the embodiments of the present invention is not limited by the following embodiments, the skilled in the art can base on the embodiments disclosed here to deduct other embodiments, all this kind of embodiments should be in the scope of the present invention.

### EMBODIMENTS

### Example 1

### The Inhibition Effect of PDPK F_{A}/GSK-3α by OPC

[Materials and Methods]

### 1. The Production, Identification and Features of Anti PDPK F_{A}/GSK-3α Antibody

PDPK F_{A}/GSK-3α amino acid sequence C-terminal 471˜483 a.a-QSTDATPTLTNSS was synthesized by Peptide Synthesizer (Model: 9050, Milligen, Bedford, Md.). According to the process Reichlin (1980) described, in order to couple the peptide onto bovine serum albumin, using glutaraldehyde as an inter-connector to connect the cysteine to NH₂-terminal. Then, the product was purified by Affinity Chromatography and neutralized by PDPK F_{A}/GSK-3α amino acid sequence C-terminal 471˜483 a.a. After the experiment, we demonstrated that this anti-PDPK F_{A}/GSK-3α antibody performs immune specificity.

### 2. Immunohistochemistry Analysis

Tissue sections (5 μm) of formalin-fixed, paraffin-embedded tissue containing tumor that showed the maximum extent of tumor cells were dewaxed in xylene and rehydrated in graded concentrations of ethanol. Endogenous peroxidase was blocked with 3% hydrogen peroxide followed by bovine serum albumin blocking for 5 minutes. The slides were next incubated with anti-PDPK F_{A}/GSK-3α antibody (2 μg/mL) diluted in 0.05 M Tris buffer, pH 7.4, at 4 for 16 hours followed by 20-minute incubation at room temperature with super enhancer (Super Sensitive™ Non-Biotin Detection System, [BioGenex, San Ramon, Calif.]), and another 30-minute incubation with polymer-HRP (Super Sensitive™) label. Immunostaining was finally developed with DAB (3-3′ diaminobenzidine tetrahydrochloride). After quenching the enzyme reaction, slides were incubated in DS-enhancer (Zymed, San Francisco, Calif.) at room temperature for five minutes to prevent the interaction between two staining system. Then, slides were incubated in CD34, vimentin, CD68 or α-SMA antibody for one hour at room temperature. After washing, slides were incubated with anti-mouse alkaline phosphatase for 30 minutes at room temperature. BCIP/NBT solution was used for visualisation of the bound antibody. Sections were counterstained with methyl green solution.

### 3. Immunocytochemistry Analysis

On average 1×10⁶ cells were centrifuged at 700 rpm for 3 minutes at room temperature (Kubota 5200, Japan), and the cell was adhered on the poly-L-lysine-coated slides. Before staining, the cytospots were fixed with 3.7% paraformaldehyde for 15 minutes and treated with 0.2% Triton X-100 for 90 seconds. Endogenous peroxidase was blocked with 3% hydrogen peroxide followed by bovine serum albumin blocking for 10 minutes. The slides were incubated with anti-PDPK FA/GSK-3α antibody (2 μg/mL) diluted in 0.05 M Tris buffer, pH 7.4, at 4 for 16 hours followed by 20-minute incubation at room temperature with super enhancer (Super Sensitive™ Non-Biotin Detection System, [BioGenex, San Ramon, Calif.]), and another 30-minute incubation with polymer-HRP (Super Sensitive™) label. Immunostaining was finally developed with DAB (3-3′ diaminobenzidine tetrahydrochloride). After quenching the enzyme reaction, slides were incubated in DS-enhancer (Zymed, San Francisco, Calif.) at room temperature for five minutes to prevent the interaction between two staining system. Then, slides were incubated with CD34, vimentin, CD68 or α-SMA antibody for one hour at room temperature. After washing, slides were incubated with anti-mouse alkaline phosphatase for 30 minutes at room temperature. BCIP/NBT solution was used for visualization of the bound antibody. Sections were counterstained with methyl green solution.

### 4. Method for Detecting Unhealthy BMC/Lethal Cell

A method for detecting unhealthy BMC/lethal cell in subject, which includes obtaining a biological sample from the subject and analyzing the PDPK F_{A}/GSK-3α expression level in BMC of the sample, wherein the aberrant accumulation of the PDPK F_{A}/GSK-3α in BMC represents the presence of unhealthy BMC in the subject.

The expression of PDPK F_{A}/GSK-3α is determined by assaying PDPK F_{A}/GSK-3α protein levels such as an immunoassay using antibodies specific for PDPK F_{A}/GSK 3α. The expression can be determined by assessing activity, protein, RNA or DNA level. The biological sample could be bone marrow, cord blood, peripheral blood, tissue sample, ascites, pleural effusion or body fluid.

### [Result]

In order to detect the unhealthy BMC in a subject, obtaining a biological sample from the subject and analyzing the PDPK F_{A}/GSK-3α expression level in BMC of the sample, wherein aberrant accumulation of PDPK F_{A}/GSK-3α expression represents the presence of unhealthy BMC in the subject.

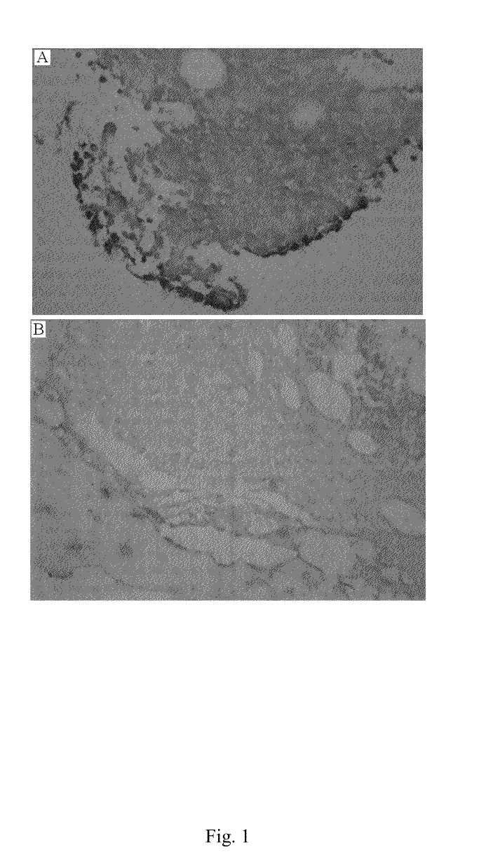

To establish the systemic role of PDPK FA/GSK-3α in bone marrow cell (BMC), an independent cohort study on bone marrow of 24 leukemia patients was performed. In a cohort study of 24 cases, 14 cases were negative and 10 cases were found to be associated with aberrant expressions of PDPK FA/GSK-3α. The immunophenotyping analysis of CD34 further revealed that a rare population of CD34+ hematopoietic stem/progenitor cells, CD34− mesenchymal stem/progenitor cells (Moioli et al, PLoS ONE, 2008, 3:e3922) associated with very strong expressions (>3+) of PDPK FA/GSK-3α, and their derivatives associated with moderate to strong expressions (2+ to 3+) of PDPK FA/GSK-3α (FIG. 1B) in contrast to the negative cases (FIG. 1A) could be frequently detected in bone marrow of those patients with progressive diseases.

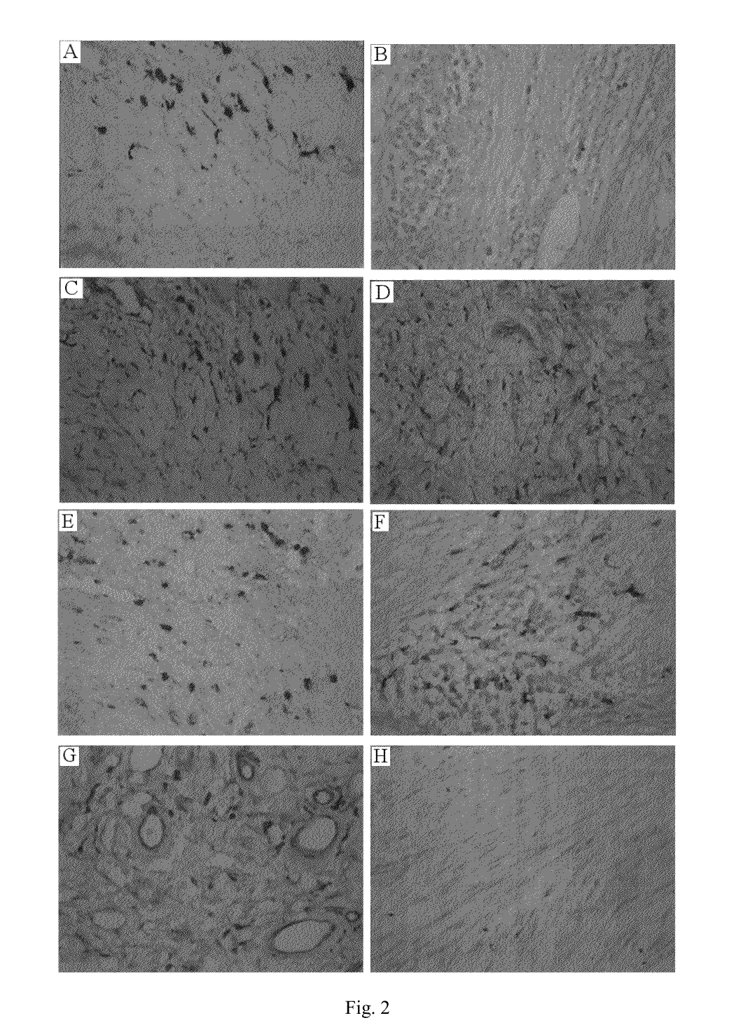

Immunophenotyping analysis of CD34, CD68, vimentin and α-smooth muscle actin (α-SMA) further revealed that a rare population of CD34+/vimentin− hematopoietic stem/progenitor cells, CD34+/vimentin+ fibrocytes, and CD34−/vimentin+ mesenchymal stem/progenitor cells (Bucala, Fibrocytes, World Scientific Pub Co Inc, 2007:1-18; Moioli et al, PLoS ONE, 2008, 3:e3922) associated with very strong expressions (>3+) of PDPK FA/GSK-3α(FIGS. 2A and C) within the stromas of incurable stage I tumors with poor prognosis even after curative resections. As described in Example I, along with a relatively large population of CD68+ macrophages (FIG. 2E), vimentin+ fibroblasts and mesenchymal cells (FIG. 2C) and α-SMA+ myofibroblasts (FIG. 2G) associated with moderate to strong expressions (2+ to 3+) of PDPK FA/GSK-3α in contrast to the negative cases with good outcome (see FIGS. 2B, D, F and H) could be detected predominantly.

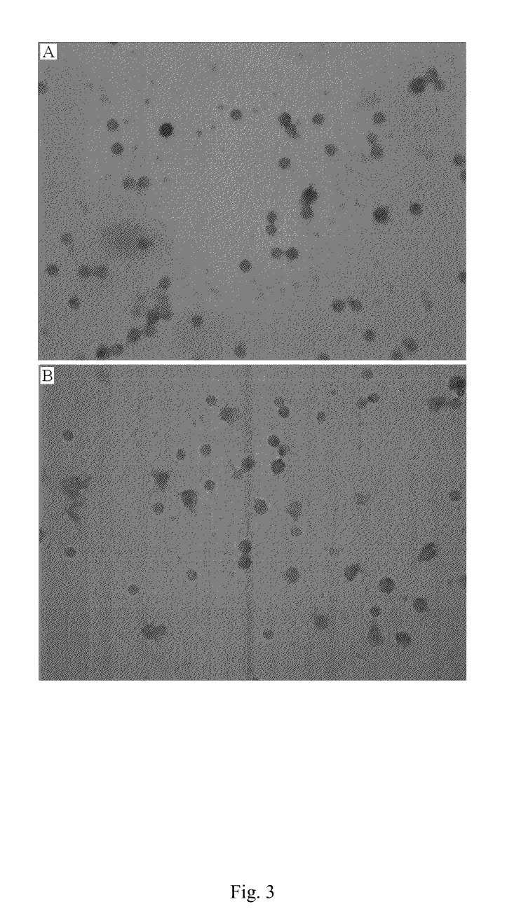

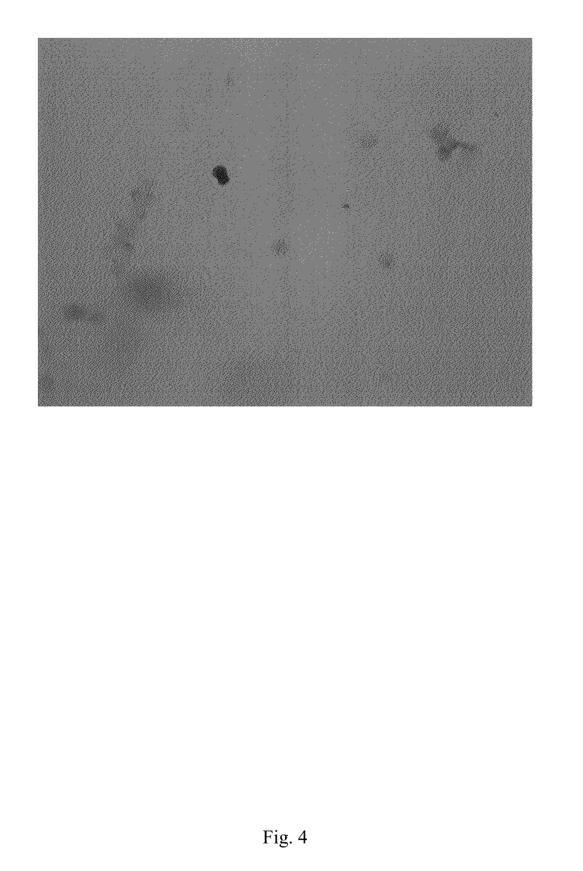

Furthermore, CD34+ fibrocyte-like cell associated with strong expression of PDPK FA/GSK-3α could be detected in peripheral blood of all the tested 3 children from 3 different families suffering from repeated cycles of inflammation (FIG. 3A and FIG. 3B). The unhealthy BDMC can be detected in cord blood from systematic unhealthy children who were suffered from Kawasaki syndrome (the symptom is immune system disorders) (FIG. 4).

In a cohort study of 367 stage I tumor cases (including all stage of bladder cancer, breast cancer, brain cancer, bile duct cancer, cervical cancer, colon cancer, endometrial cancer, esophageal cancer, stomach cancer, liver cancer, lung cancer, nasopharyngeal carcinoma, oral cancer, ovary cancer, pancreatic cancer, prostate cancer, kidney cancer, leukemia and lymphoma), 240 cases were negative, and 127 cases were related to PDPK F_{A}/GSK-3α abnormal expression.

In order to detect the lethal cell in a subject, obtaining a biological sample from the subject and analyzing the PDPK F_{A}/GSK-3α expression in BMDSC of the sample, wherein aberrant accumulation of PDPK F_{A}/GSK-3α expression in BMDSC represents the presence of lethal cell in the subject.

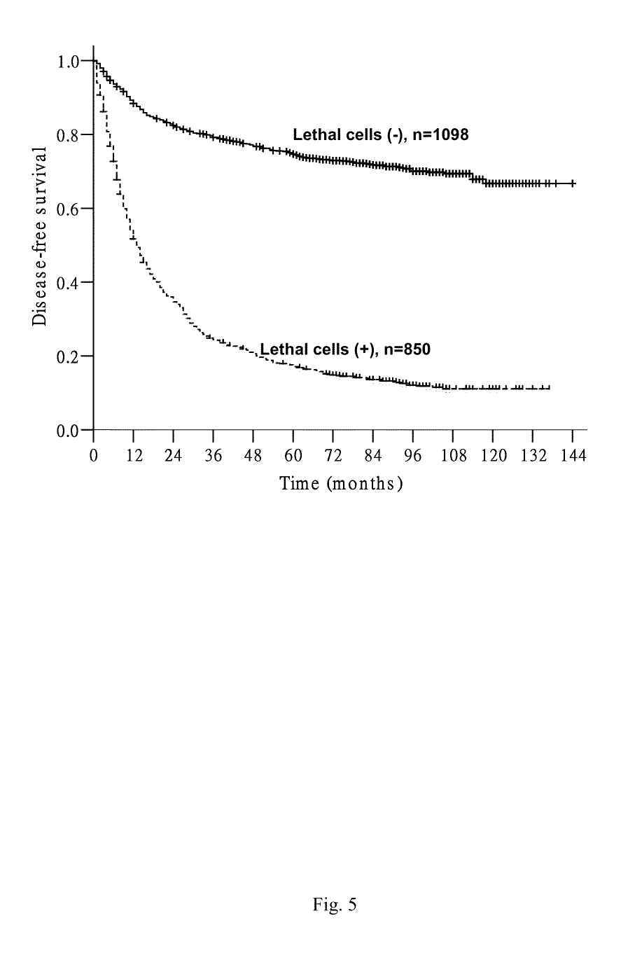

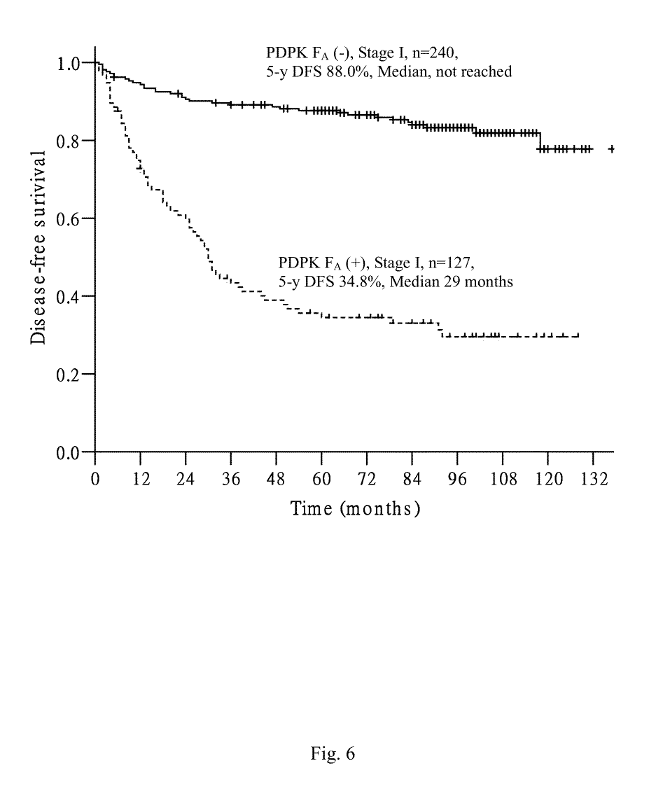

In a cohort study of 1948 tumor cases (including all stage of bladder cancer, breast cancer, brain cancer, bile duct cancer, cervical cancer, colon cancer, endometrial cancer, esophageal cancer, stomach cancer, liver cancer, lung cancer, nasopharyngeal carcinoma, oral cancer, ovary cancer, pancreatic cancer, prostate cancer, kidney cancer, leukemia and lymphoma), 1098 cases were negative of lethal cell, and 850 cases were related to PDPK F_{A}/GSK-3α aberrant accumulation lethal cell (FIG. 5). When a patient has abnormal expression of signal molecules in the tumor matrix, he/she is more likely to have worse prognosis (FIG. 6).

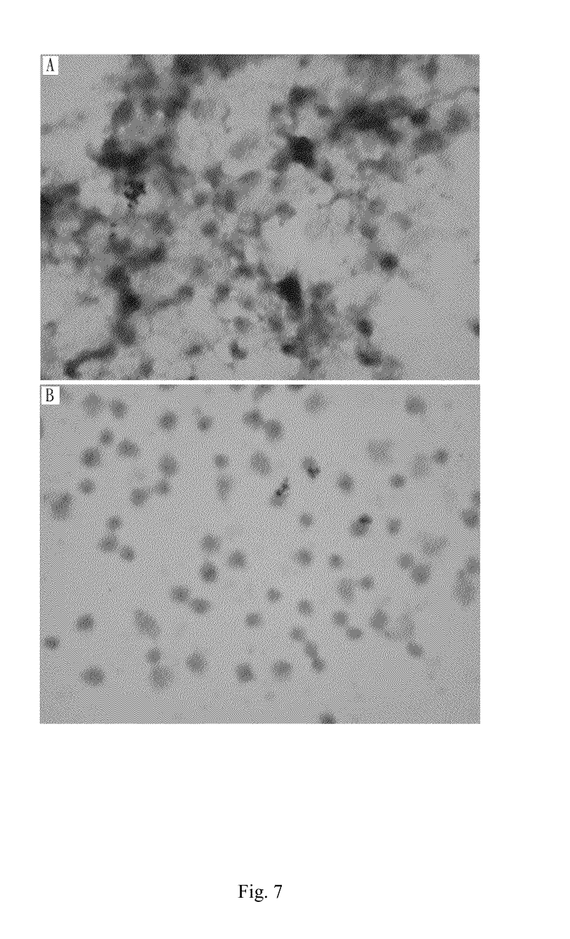

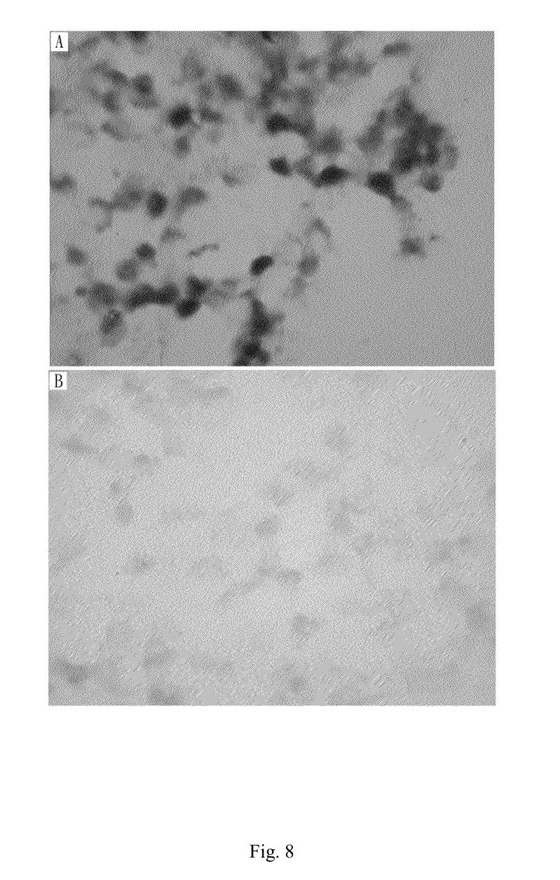

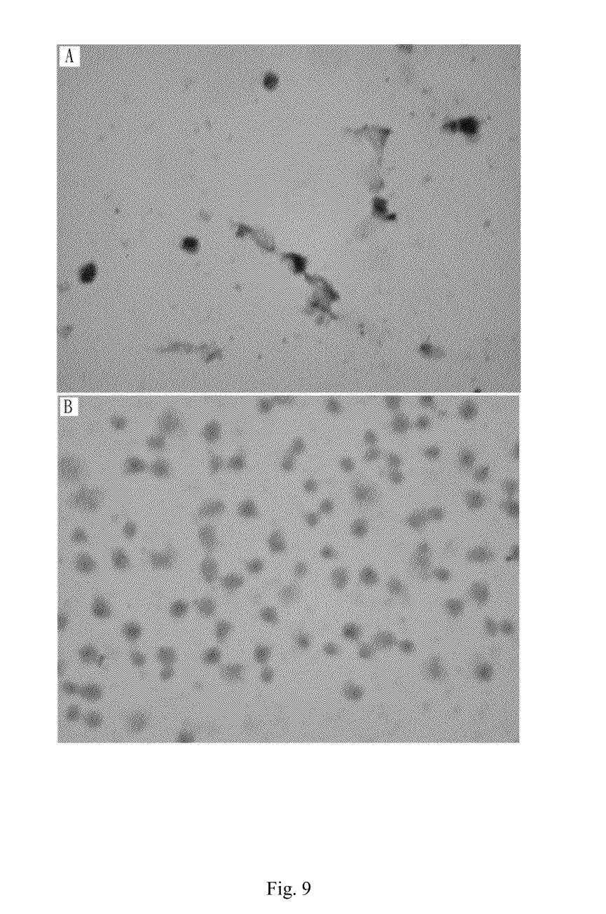

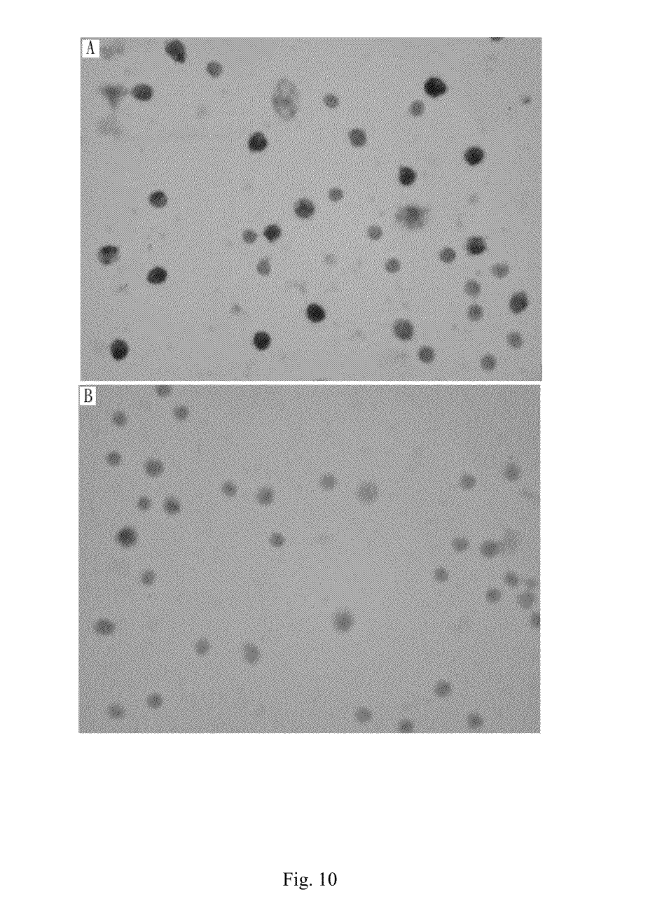

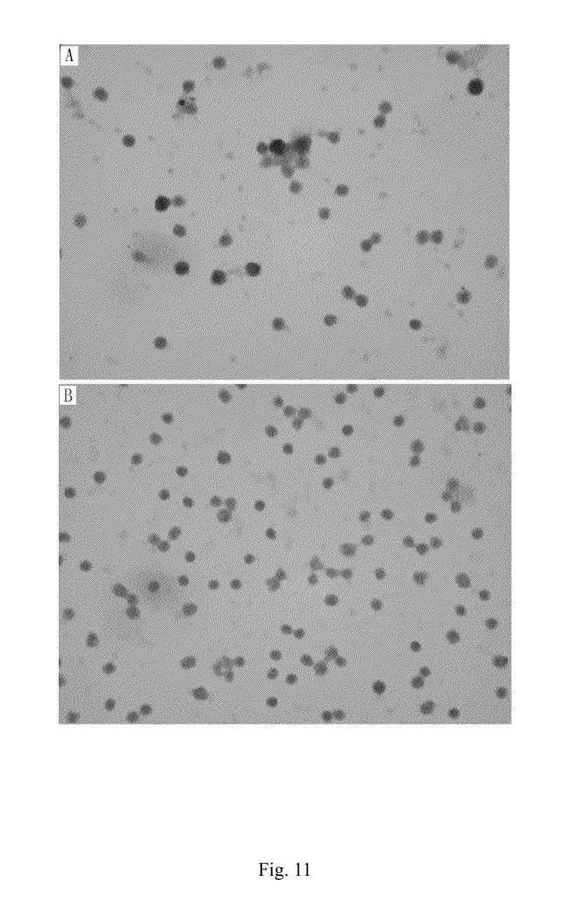

Before and after using OPC, the PDPK F_{A}/GSK-3α expression conditions in patients were analyzed by immunochemistry. Analyzing the following samples: acute leukemia (before OPC: FIG. 7A, after OPC: FIG. 7B), lymphoma (before OPC: FIG. 8A, after OPC: FIG. 8B), kidney cancer (before OPC: FIG. 9A, after OPC: FIG. 9B), uterine sarcoma (before OPC: FIG. 10A, after OPC: FIG. 10B) and children with systematic unhealthy from cancer family (before OPC: FIG. 11A, after OPC: FIG. 11B), the result indicates that the samples of patients show PDPK F_{A}/GSK-3α positive result (red-brown) before using OPC, and that the samples of patients show PDPK F_{A}/GSK-3α negative result after using OPC.

In terms of the above, the present invention provides methods of cancer prevention, disease predisposition prevention, metastasis prevention and vicious cycle prevention by OPC induced PDPK F_{A}/GSK-3α inhibition.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. A composition for inhibiting proline-directed protein kinase F_{A}/glycogen synthase kinase 3α (PDPK FA/GSK-3α) which composition comprises an oligomer procyanidins (OPCs).

2. The composition of claim 1, wherein the oligomer procyanidins is extracted by grape, grape skin, grape seed, pine bark, bark of lemon tree, peanuts, berries, sorghum, apple, coco bean, cherry, strawberry, hops, salix, cinnamon, tea leaf, Wax Tree, Pinto Bean, red kidney beans, hazelnut, or walnut.

3. The composition of claim 1, further comprising a pharmaceutically acceptable carrier.

4. The composition of claim 3, wherein the pharmaceutical acceptable carrier is excipient, diluents, thickeners, filler, binder, disintegrants, lubricant, oil or non-oil base, surfactant, suspending agent, gelling agent, adjuvant, anti-corrosive agent, anti-oxidant, stabilizer, coloring agent or flavor.

5. A method for treating an unhealthy state in a subject through inhibiting PDPK FA/GSK-3α expression by administering an effective amount of OPC.

6. The method of claim 5, wherein the unhealthy state is a human who is free of diagnosable cancer and has an unhealthy BMC.

7. The method of claim 5, wherein the unhealthy state is a human who is free of diagnosable cancer and has a vicious cycle.

8. The method of claim 6, wherein the unhealthy BMC is a BMC associated with an aberrant intracellular accumulation of PDPK FA/GSK-3α.

9. The method of claim 8, wherein the BMC is selected from the group consisting of stem cell, bone marrow derived cell (BMDC), mesenchymal stem cell (MSC), hematopoietic stem and progenitor cell (HSPC), cancer stem cell (CSC), fibrocyte, macrophage, fibroblast, myoblasts, mesenchymal cell, and the cell analogue.

10. The method of claim 8, wherein the aberrant intracellular accumulation of PDPK FA/GSK-3α expression is in cytoplasmic and/or nuclear expression of PDPK FA/GSK-3α protein, mRNA or DNA level.

11. The method of claim 7, wherein the vicious cycle is stresses and/or inflammations responses associated with an aberrant accumulation of PDPK FA/GSK-3α.

12. A method for treating a cancer patient through inhibiting PDPK FA/GSK-3α expression by administering an effective amount of OPC.

13. The method of claim 12, wherein the cancer patient has a lethal cell.

14. The method of claim 12, wherein the cancer patient has a lethal system.

15. The method of claim 13, wherein the lethal cell is a BMDSC associated with an aberrant intracellular accumulation of PDPK FA/GSK-3α.

16. The method of claim 14, wherein the lethal system is a signaling interplay network associated with aberrant expression of PDPK FA/GSK-3α in marker cell.

17. The method of claim 16, wherein the signaling interplay network is selected from the group consisting of epithelial-mesenchymal transition (EMT) and tumor-stroma coevolutional communication (TSCC).

18. The method of claim 16, wherein the marker cell is selected from the group consisting of bone marrow-derived cell (BMDC), carcinoma-associated fibroblasts (CAF), circulating tumor cell (CTC), circulating cancer stem cell (CTSC), epidermal tumor cell (ETC), epidermal tumor stem cell (ETSC), cancer stem cell (CSC), tumor proliferate cell (TPC), hematopoietic stem and progenitor cell (HSPC), mesenchymal stem cell (MSC), mesenchymal tumor cell (MTC), mesenchymal tumor stem cell (MTSC), tumor-associated macrophage (TAM), and myeloid cell.

19. The method of claim 12, wherein the cancer is selected from a group consisting of bladder cancer, breast cancer, brain cancer, cancer of biliary duct, uterine cancer, esophageal cancer, stomach cancer, hepatic cancer, lung cancer, oral cancer, ovarian cancer, pancreatic cancer, prostate cancer, kidney cancer, leukemia and lymphoma.
